(19) United States
(12) Patent Application Publication
(10) Pub. No.: US 2021/0052358 A1
(43) Pub. Date: Feb. 25, 2021

(54) TEETH WHITENING PRODUCT CONTAINING PIEZOELECTRIC MATERIAL

(71) Applicant: Nanjing University of Science & Technology, Nanjing (CN)

(72) Inventors: Yaojin WANG, Nanjing (CN); Yang WANG, Nanjing (CN); Nana YANG, Nanjing (CN); Xuan CHEN, Nanjing (CN); Xinrong WEN, Nanjing (CN); Shuhao WANG, Nanjing (CN); Hanzhou WU, Nanjing (CN); Bin YANG, Nanjing (CN)

(21) Appl. No.: 16/928,489

(22) Filed: Jul. 14, 2020

(30) Foreign Application Priority Data
Aug. 19, 2019 (CN) 201910762356.1

(51) Int. Cl. A61C 19/06; A61C 17/34; A61K 8/29; A61K 8/27; A61K 8/81; A61K 8/19; A61Q 11/00

(52) U.S. Cl. CPC A61C 19/066; A61C 17/3481; A61K 8/29; A61K 8/27; A61C 2204/00; A61K 8/19; A61Q 11/00; A61C 2201/00; A61K 8/8123

(57) ABSTRACT

The present invention discloses a teeth whitening product containing a piezoelectric material. The present invention uses a piezoelectric material with piezoelectric effect as a whitening ingredient of the teeth whitening product. The teeth whitening product is applied to teeth and exerts pressure on the tooth surface to activate piezoelectric properties of the piezoelectric material. Thus, abundant reactive species are produced to degrade teeth stains, achieving teeth whitening. The teeth whitening product of the invention is safe and nontoxic; a teeth whitening method is easy to operate, does not cause damage to oral soft tissues, enamel layers on the tooth surface, and the tooth structure, and obtains a long-lasting teeth whitening effect.

### CROSS-REFERENCE TO RELATED APPLICATIONS

This application claims priority to CN Patent Application No. 201910762356.1, filed Aug. 19, 2019, the entire contents of which are hereby incorporated herein by reference.

### TECHNICAL FIELD

The present invention relates to a teeth whitening product containing a piezoelectric material, and belongs to the technical field of teeth whitening.

### BACKGROUND

Nowadays, there are two major teeth whitening methods. One method is to use abradants to manage tooth discoloration and teeth stains. However, abradants have a limited effect on teeth whitening, and result in damaged enamel layer on the tooth surface. Further, such damage may boost the odds of repeated tooth discoloration. The other is to use whitening active substances. A whitening agent is applied on teeth for a spell, and active substances act on teeth to achieve the purpose of whitening eventually. Main components of common whitening agents are peroxides. When high-concentration hydrogen peroxide is used, a stronger whitening effect can be observed. That is, as hydrogen peroxide concentration rises, there is an increase in the degree of infiltration into teeth due to concentration difference, and the amount of reacting hydrogen peroxide rises, thereby improving pigmentary degradation, i.e., teeth whitening effect.

However, no matter whether whitening agents (e.g., 30% H₂O₂, carbamide peroxide, sodium perborate, etc.) or cold light teeth whitening kits are used, main whitening ingredient thereof is still hydrogen peroxide. At high concentrations, hydrogen peroxide may damage human skin and mucosa; also, due to a difference in hydrogen peroxide concentration, whitening agents may erode both enamel and dentin at different levels, and even injure dental pulps, causing some irreparable damage to teeth. These methods can bleach and luster teeth in the short run, but over time, tooth discoloration appears more easily and even teeth may turn gray (i.e., nonvital teeth) due to damaged enamel layer on the tooth surface. Furthermore, because of high cost of an apparatus for bleaching teeth, patients need to pay a high price for each tooth bleaching; due to a complex procedure during operation, special work conditions and stricter working environment are required.

### SUMMARY

The present invention provides an efficient, simple, economic, nondestructive and bleach-free teeth whitening product containing a piezoelectric material. Degradation of teeth stains is achieved by means of the piezoelectric effect of the piezoelectric material.

The piezoelectric material of the present invention is a material with piezoelectric effect, and may be one of BaTiO₃, GaN, ZnSnO₃, (Na,K)—NbO₃, ZnO, lead zirconate titanate, lead magnesium niobate, polyvinylidene fluoride and polyvinydene fluoride, or a complex or composition of a plurality of materials, e.g., PMN-PT.

The piezoelectric material of the present invention is preferably a poled piezoelectric material. The poled piezoelectric material has a better teeth whitening effect than an unpoled one.

The teeth whitening product containing a piezoelectric material provided by the present invention may be conventional in the art, e.g., teeth whitening agent, teeth whitening apparatus, etc.

The teeth whitening agent of the present invention may be a piezoelectric material or a teeth whitening composition containing a piezoelectric material. The teeth whitening agent may be given in various forms, which may be powdery, liquid, colloidal, gelatinous, or pasty.

The teeth whitening composition containing a piezoelectric material provided by the present invention may be toothpaste, dentifrice, chewing gum, a chew, a gel, or a tooth cleanser, any of which contains piezoelectric material.

The teeth whitening apparatus of the present invention is a teeth whitening apparatus prepared with a piezoelectric material. The teeth whitening apparatus is given in various forms, which may be a toothbrush with bristles made of piezoelectric material, or a brace or denture prepared with a piezoelectric material.

The present invention further provides a teeth whitening method, where a teeth whitening product containing a piezoelectric material is applied to the tooth surface and exerts pressure on the tooth surface to achieve teeth whitening.

Specifically, the teeth whitening method of the present invention achieves teeth whitening by applying or spraying the teeth whitening agent to the tooth surface and exerting pressure on the tooth surface. A method for exerting pressure on the tooth surface may be vibration or friction, e.g., by means of occlusion or using such dental care products as toothbrush. For example, repeated action of tooth brushing provides the piezoelectric material with continuous pressure change and activates piezoelectric properties of the piezoelectric material. Thus, abundant reactive species are produced to degrade teeth stains, achieving teeth whitening.

Specifically, the teeth whitening method of the present invention achieves teeth whitening by exposing the teeth whitening apparatus to teeth and exerting pressure on the tooth surface. A method for exerting pressure on the tooth surface may be vibration or friction, e.g., by means of occlusion or using a toothbrush to brush teeth repeatedly; alternatively, external pressure device provides the piezoelectric material with continuous pressure change and activates piezoelectric properties of the piezoelectric material. Thus, abundant reactive species are produced to degrade teeth stains, achieving teeth whitening.

In view of the teeth whitening method of the present invention, the efficiency of the piezoelectric material whitening teeth primarily depends on how many excitation signals the piezoelectric material receives. Therefore, use of high vibration frequency electric toothbrushes or other devices has more significant whitening effect than use of regular toothbrushes.

Compared with the prior art, the present invention has the following advantages:

The present invention uses a piezoelectric material with piezoelectric effect as a whitening ingredient of the teeth whitening product. The teeth whitening product is applied to teeth and exerts pressure on the tooth surface to activate piezoelectric properties of the piezoelectric material. Thus, abundant reactive speciesare produced to degrade teeth stains, achieving teeth whitening. The teeth whitening method of the invention is easy to operate, does not need additional equipment and professional operators, does not damage the tooth structure, and obtains a long-lasting teeth whitening effect.

### BRIEF DESCRIPTION OF DRAWINGS

FIG. 1 is a schematic diagram showing the catalytic effect of a piezoelectric material.

FIG. 2 is a schematic diagram showing a mechanism underlying piezo-catalytic degradation of teeth stains.

FIG. 3 is a schematic diagram showing a mechanism underlying catalytic degradation of teeth stains by a tooth coating retainer containing a piezoelectric material.

FIG. 4 illustrates experimental results of ultrasonic degradation of rhodamine B by poled and unpoled barium titanate in Example 1.

FIG. 5 illustrates pre- and post-sonicated changes in vinegar-stained teeth in the absence and presence of barium titanate in Example 2.

FIG. 6 illustrates biological toxicity testing results of barium titanate powders and 15% H₂O₂.

FIG. 7 is a scanning electron micrograph of the tooth surface after whitening with barium titanate and 30% H₂O₂ for the same time period.

### DETAILED DESCRIPTION

The present invention is described in more detail below with reference to the embodiments and accompanying drawings.

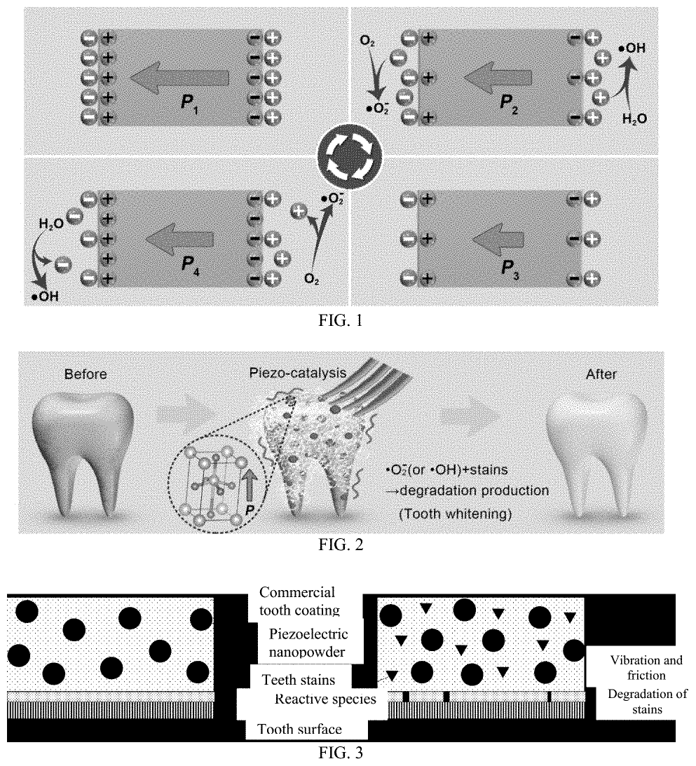

FIG. 1 is a schematic diagram showing the catalytic effect of a piezoelectric material. As shown in FIG. 1, unlike charges exist on the surface of the piezoelectric material because of its own polarity; when an external force exerted on the piezoelectric material changes, unlike charges will be released from the surface of the piezoelectric material. In an aqueous solution, those unlike charges released will bind to molecules in the water to form reactive species, which can degrade teeth stains. When the external force stops changing, a new electrostatic equilibrium is formed on the surface of the piezoelectric material. When the external force changes again, the piezoelectric material absorbs unlike charges from the aqueous solution, and then charges retained in the water continue to react with water molecules to form reactive species. Therefore, the piezoelectric material can be used for catalytic degradation as long as a continuous pressure change is exerted on the piezoelectric material.

FIG. 2 is a schematic diagram showing a mechanism underlying catalytic degradation of teeth stains by a piezoelectric material through the piezoelectric effect. In order to achieve a catalytic whitening effect, the piezoelectric material is applied to the tooth surface and activates the piezoelectric effect by means of stimulation, e.g., vibration and friction.

FIG. 3 is a schematic diagram showing a mechanism underlying catalytic degradation of teeth stains by a tooth coating retainer containing a piezoelectric material. As shown in FIG. 3, piezoelectric powders or a teeth whitening composition containing piezoelectric powders is applied on the tooth surface; a toothbrush is used to brush teeth repeatedly, providing the piezoelectric material with continuous pressure change and activating piezoelectric properties of the piezoelectric material. Thus, abundant reactive species are produced to degrade teeth stains, achieving teeth whitening.

### Example 1

In the example, barium titanate is used as an example of piezoelectric materials to demonstrate that piezoelectric materials have a catalytic effect under ultrasound.

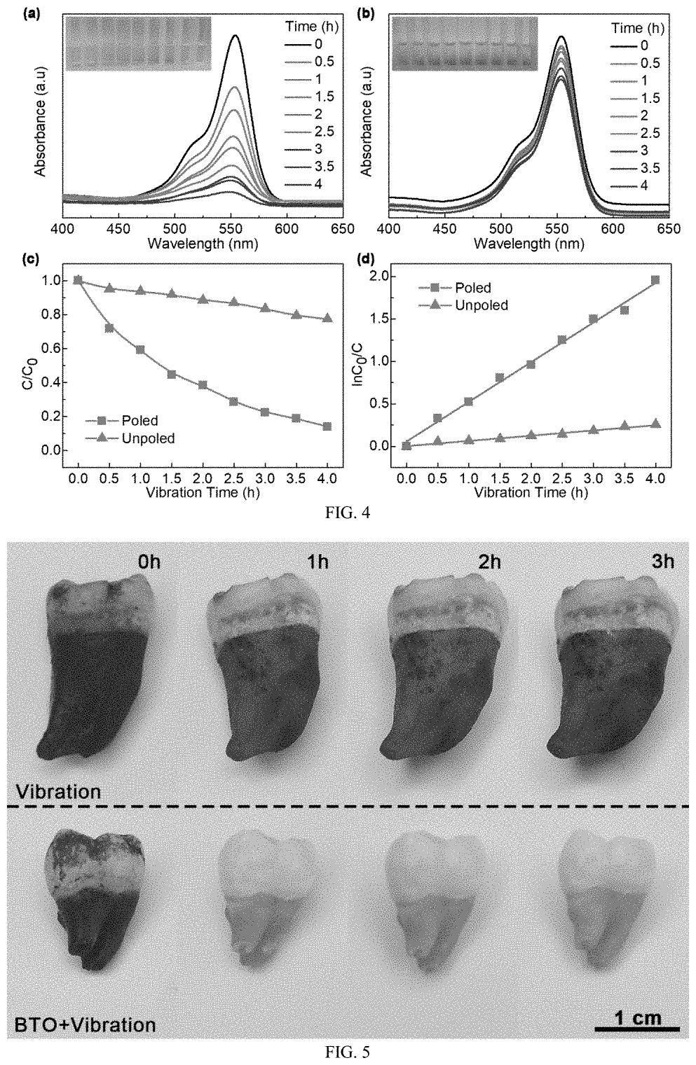

Poled and unpoled barium titanate was placed and sonicated in 5 mg/L rhodamine B solution, respectively. FIG. 4(a) shows a spectrogram of absorbance of rhodamine B as a function of time after addition of poled barium titanate. FIG. 4(b) shows a spectrogram of absorbance of rhodamine B as a function of time after addition of unpoled barium titanate, revealing a significant increase in catalytic rate of the poled barium titanate. In contrast, from FIGS. 4(c) and 4(d), poled barium titanate has a significantly better catalytic effect than unpoled one.

As indicated by the example, regardless of polarization, barium titanate can realize the degradation of organic compounds under ultrasound, but poled barium titanate shows a more significant piezoelectric response; therefore, the catalytic rate is faster. The example demonstrates the feasibility of piezoelectric materials in the application of teeth whitening.

### Example 2

In order to simulate the process of vibration whitening, vinegar-stained teeth were placed in a suspension with 1 mg/ml piezoelectric barium titanate powder, and sonicated for 3 h at 40 W at 1 kHz. Barium titanate powder was poled, with a particle size of ˜130 nm. After sonication for 3 h, the teeth were whitened significantly.

As shown in FIG. 5, the top teeth reveal a color change 3 h after sonication in clear water; the bottom teeth reveal a color change 3 h after sonication in the presence of piezoelectric barium titanate powder. Visibly, the teeth are whitened significantly after addition of barium titanate.

### Example 3

Teeth stained with a mixed solution of black tea, wine, fruity beverages, and blueberry juice were placed and sonicated in a suspension with Mn-PMN-PT. In the suspension, a concentration of piezoelectric powder was 1 mg/ml, and the Mn-PMN-PT was poled, with a particle size of ˜1 μm. After sonication for 3 h at 40 W at 1 kHz, the teeth were whitened significantly.

### Example 4

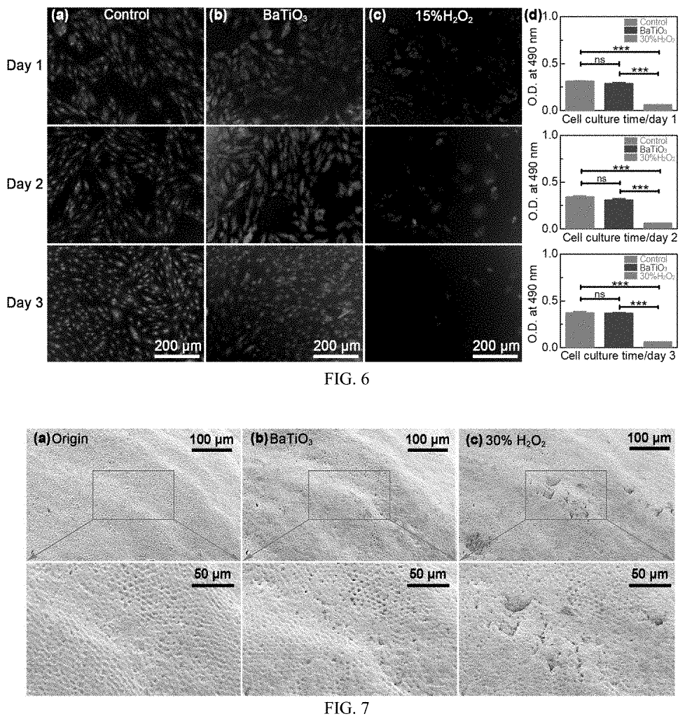

A biological toxicity testing was conducted on barium titanate powders using rat thoracic aortic smooth muscle cells. Different additives were charged into cell culture media. After culture for a period of time, biological statistics was conducted. Results are shown in FIG. 6. FIG. 6(a) illustrates a cell count in a standard medium as a function of cell culture time; FIGS. 6(b) and 6(c) illustrate results after addition of barium titanate powders (final concentration: 1 mg/mL) and 30% H₂O₂ (final concentration: 15%) in a culture medium, respectively; FIG. 6(d) illustrates mathematical statistics of results of every day. As can be seen, addition of barium titanate in the standard medium has no effect on cell growth and proliferation; on the contrary, almost all cells in the medium die after addition of H₂O₂, suggesting that barium titanate has good biocompatibility.

In view of the teeth whitening method of the present invention, main active component is a piezoelectric material, and there is no risk of damage to teeth, skin, nerves, and mucosae in the process of whitening with peroxides. FIG. 6 illustrates biological toxicity testing results of barium titanate powders and 30% H₂O₂. As illustrated in FIG. 6, by comparison of piezoelectric barium titanate powders and 30% H₂O₂, piezoelectric barium titanate powders are significantly less biotoxic than 30% H₂O₂; also, barium titanate powders do not damage the tooth surface, but 30% H₂O₂ causes non-ignorable damage to the enamel on the tooth surface. FIG. 7 is a scanning electron micrograph of the tooth surface after whitening with barium titanate and 30% H₂O₂ for the same time period. As can be seen, H₂O₂ damages the tooth surface seriously, but after whitening with barium titanate, there is no damage on the tooth surface, and the morphology is almost the same as original teeth.

In conclusion, with the piezoelectric response of the piezoelectric material, use of the teeth whitening product containing a piezoelectric material can achieve teeth whitening on the basis of no damage to the teeth and no irreparable teeth injury. The piezoelectric material is applied to the tooth surface and activates the piezoelectric effect by means of stimulation, e.g., vibration and friction, achieving a catalytic whitening effect. As shown in FIG. 2, piezoelectric powders or a teeth whitening composition containing piezoelectric powders is applied on the tooth surface; a toothbrush is used to brush teeth repeatedly, providing the piezoelectric material with continuous pressure changes and activating piezoelectric properties of the piezoelectric material. Thus, abundant reactive species are produced to degrade teeth stains, achieving teeth whitening.

The teeth whitening method of the present invention may provide a plurality of examples. Examples are as follows. (1) When the piezoelectric material is combined with commercial toothpaste, piezo-catalytic teeth whitening can be achieved by means of the vibration generated during tooth brushing, and whitening effect will be better if an electric toothbrush with higher excitation intensity is used. (2) The piezoelectric material is made into fibers to serve as bristles of a toothbrush. When the toothbrush is used, bent bristles can trigger the piezoelectric effect to achieve piezo-catalytic teeth whitening. (3) When the piezoelectric material is combined with commercial chewing gum, piezo-catalytic teeth whitening can be achieved by means of the vibration and friction generated during chewing. (4) When the piezoelectric material is added to oral care or orthodontic appliances, e.g., gutta-percha, similar to the chewing gum, piezo-catalytic teeth whitening can be achieved by means of vibration and friction generated during chewing; also, when the piezoelectric material is added to such appliances as brace and denture, piezo-catalytic teeth whitening can be achieved by means of the friction between brace and denture during occlusion in the process of orthodontics. (5) When the piezoelectric material is directly added to a teeth whitening apparatus, piezo-catalytic teeth whitening can be achieved by means of extra stimulation. In above examples, tooth-brushing, chewing of such food products as chewing gum, and orthodontic appliances are long-term daily activities. Combination of teeth whitening with daily activities in daily life can achieve teeth whitening.

What is claimed is:

1. A teeth whitening product containing a piezoelectric material.

2. The teeth whitening product according to claim 1, wherein the piezoelectric material is a material with piezoelectric effect, selected from one of BaTiO₃, GaN, ZnSnO₃, (Na,K)—NbO₃, ZnO, lead zirconate titanate, lead magnesium niobate, polyvinylidene fluoride, and polyvinylidene fluoride, or a complex or composition of a plurality of materials, e.g., PMN-PT.

3. The teeth whitening product according to claim 1, wherein the piezoelectric material is a poled piezoelectric material.

4. The teeth whitening product according to claim 1, wherein the product may be a teeth whitening agent or a teeth whitening apparatus.

5. The teeth whitening product according to claim 4, wherein the teeth whitening agent may be a piezoelectric material or a teeth whitening composition containing a piezoelectric material; the teeth whitening agent may be given in powdery, liquid, colloidal, gelatinous, or pasty form.

6. The teeth whitening product according to claim 5, wherein the teeth whitening composition containing a piezoelectric material may be toothpaste, dentifrice, chewing gum, a chew, a gel, or a tooth cleanser, any of which contains piezoelectric material.

7. The teeth whitening product according to claim 4, wherein the teeth whitening apparatus is a teeth whitening apparatus prepared with a piezoelectric material; the teeth whitening apparatus is a toothbrush with bristles made of piezoelectric material, or a brace or denture prepared with a piezoelectric material.

8. A teeth whitening method, wherein the teeth whitening product according to claim 1 is applied to the tooth surface and exerts pressure on the tooth surface.

9. A teeth whitening method, wherein the teeth whitening product according to claim 2 is applied to the tooth surface and exerts pressure on the tooth surface.

10. A teeth whitening method, wherein the teeth whitening product according to claim 3 is applied to the tooth surface and exerts pressure on the tooth surface.

11. A teeth whitening method, wherein the teeth whitening product according to claim 4 is applied to the tooth surface and exerts pressure on the tooth surface.

12. A teeth whitening method, wherein the teeth whitening product according to claim 5 is applied to the tooth surface and exerts pressure on the tooth surface.

13. A teeth whitening method, wherein the teeth whitening product according to claim 6 is applied to the tooth surface and exerts pressure on the tooth surface.

14. A teeth whitening method, wherein the teeth whitening product according to claim 7 is applied to the tooth surface and exerts pressure on the tooth surface.

15. The teeth whitening method according to claim 8, wherein a teeth whitening agent is applied or sprayed to the tooth surface and exerts pressure on the tooth surface; alternatively, a teeth whitening apparatus is applied to teeth and exerts pressure on the tooth surface; a method for exerting pressure on the tooth surface is vibration or friction.

16. The teeth whitening method according to claim 9, wherein a teeth whitening agent is applied or sprayed to the tooth surface and exerts pressure on the tooth surface; alternatively, a teeth whitening apparatus is applied to teeth and exerts pressure on the tooth surface; a method for exerting pressure on the tooth surface is vibration or friction.

17. The teeth whitening method according to claim 10, wherein a teeth whitening agent is applied or sprayed to the tooth surface and exerts pressure on the tooth surface; alternatively, a teeth whitening apparatus is applied to teeth and exerts pressure on the tooth surface; a method for exerting pressure on the tooth surface is vibration or friction.

18. The teeth whitening method according to claim 11, wherein a teeth whitening agent is applied or sprayed to the tooth surface and exerts pressure on the tooth surface; alternatively, a teeth whitening apparatus is applied to teeth and exerts pressure on the tooth surface; a method for exerting pressure on the tooth surface is vibration or friction.

19. The teeth whitening method according to claim 12, wherein a teeth whitening agent is applied or sprayed to the tooth surface and exerts pressure on the tooth surface; alternatively, a teeth whitening apparatus is applied to teeth and exerts pressure on the tooth surface; a method for exerting pressure on the tooth surface is vibration or friction.

20. The teeth whitening method according to claim 15, wherein the teeth whitening apparatus is a high vibration frequency electric toothbrush or teeth whitening apparatus.
